(19) United States
(12) Patent Application Publication
(10) Pub. No.: US 2013/0047273 A1
(43) Pub. Date: Feb. 21, 2013

(54) Genetically altered animal specimen and related methods

(71) Applicants: Rameshwar K. Sharma, Maple Glen, PA (US); Teresa Duda, Horsham, PA (US)

(72) Inventors: Rameshwar K. Sharma, Maple Glen, PA (US); Teresa Duda, Horsham, PA (US)

(21) Appl. No.: 13/136,945

(22) Filed: Aug. 16, 2011

(51) Int. Cl. A01K 67/027

(52) U.S. Cl. 800/9; 800/21

(57) ABSTRACT

A genetically altered animal specimen is provided by a process comprising: identifying a gene that is desired to be altered, disrupting the gene in a gene carrier to thereby create a new DNA fragment; inserting the new DNA fragment into an embryonic cell, injecting the embryonic cell which exhibits the desired genetic alteration into an embryo, inserting the embryo into a uterus of a carrier whereby the carrier's offspring shall exhibit the desired genetic alteration, and the offspring is the genetically altered animal specimen, in this case the neurocalcin δ gene knockout mouse model.

### BACKGROUND OF THE INVENTION

1. Field of the Invention

The present invention relates to a genetically altered animal specimen and more specifically, a method of creating a new animal specimen without the neurocalcin δ gene comprising: creating a new animal specimen that is a mouse without a neurocalcin δ gene and a design a construction to alter or eliminate the neurocalcin δ gene.

2. Description of the Related Art

Neurocalcin δ protein has been identified and studied in several areas of pharmacology and biology. These areas relate to methods for preparing inner ester derivatives for pharmaceutical compositions used to treat disorder of the nervous system.

Neurocalcin δ has also been identified and studied in the diagnosis and/or treatment of various ailments and diseases including, but not limited to heart failure, Parkinson's disease, and Alzheimer's disease. It has also been related to incorporating foreign protein segments having medically or commercially useful biological function into surface proteins of viruses. The protein has also been noted in methods for promoting regeneration of nerve tissues.

Neurocalcin δ has also been referenced in methods for delivering neuropeptides, amongst various other biological chemicals, through or across the blood brain barrier. It has also been used in exposing cells to an electric field for the detection of various kinds of cellular pathology. It has also been referenced in genetically altering bacteria in order to target proteins and binding domains. Removal of the neurocalcin δ gene in animal specimen has directly or indirectly caused the death of such animal specimen (preventing scientists and doctors from studying a specimen with a genetically removed neurocalcin δ gene).

The present invention relates to genetically altering an animal specimen to eliminate the neurocalcin δ gene and related method of creating animal specimens without the neurocalcin δ gene. The present invention's process allows for offspring to be created that survive so that they can be studied in the hopes of developing methods to treat various types of biological ailments, diseases and disorders.

### SUMMARY OF THE INVENTION

In one embodiment, the present invention relates to a genetically altered animal specimen created by a process comprising: identifying a gene that is desired to be altered; disrupting the gene in a gene carrier to thereby create a new DNA fragment; inserting the new DNA fragment into an embryonic cell; injecting the embryonic cell which exhibits the desired genetic alteration into an embryo; and inserting the embryo into a uterus of a carrier whereby the carrier's offspring shall exhibit the desired genetic alteration, and the offspring is a genetically altered animal specimen.

In another embodiment, the specimen is without the neurocalcin δ gene. In yet another embodiment, the disruption step is performed using a construction to disrupt the gene and create the DNA fragment. In still another embodiment, the insertion of the DNA fragment into the embryonic cell utilizes electroporation.

In still yet another embodiment, the process further comprises: mating female of the offspring from the embryo which exhibits the desired genetic alteration with male specimen with normal genetic makeup.

In a further embodiment, the process further comprises: mating male and female offspring which exhibit the desired genetic alterations from different mothers to thereby create a colony of specimens exhibiting the desired genetic alterations.

In another further embodiment, the animal is a mouse. In yet a further embodiment, the desired genetic alteration is a specimen without the neurocalcin δ gene. In still a further embodiment, the animal specimen exhibits characteristics comprising: the malfunction of sensory neurons; malfunction of normal fertility; learning disabilities; loss of memory; degeneration of neurons in the brain; said characteristics of the new specimen shall be identifiable based on a protein marker for the neurocalcin δ gene.

In still yet a further embodiment, the present invention relates to a genetically altered specimen created by a method comprising: eliminating the neurocalcin δ gene thereby having: malfunction of sensory neurons; malfunction of normal fertility; learning disabilities; loss of memory; degeneration of neurons in the brain; and a protein marker for the neurocalcin δ gene identifying said characteristics of the new specimen.

In one embodiment, the specimen is an animal. In another embodiment, the specimen is a mammal. In yet another embodiment, the specimen is a mouse.

In a further embodiment, the present invention relates to a process of creating a genetically altered specimen, and the process comprises: altering a selected gene in a gene carrier to thereby create a desired DNA fragment; inserting the desired DNA fragment into an embryonic cell; introducing said embryonic cell which exhibits the desired genetic alteration into an embryo; and impregnating the carrier with the embryo whereby an offspring of the carrier is the genetically altered specimen.

In another further embodiment, the specimen is without the neurocalcin δ gene. In yet another further embodiment, the alteration stage is performed using a construction to disrupt the gene and create the DNA fragment. In still another further embodiment, the insertion of the DNA fragment into the embryonic cell utilizes electroporation.

In still yet another further embodiment, the female of the offspring from the embryo which exhibits the desired genetic alteration is mated with male specimen with normal genetic makeup.

In another embodiment, the male and female offspring which exhibit said desired genetic alterations from different mothers are mated to thereby create a colony of specimens exhibiting said desired genetic alterations.

In a further embodiment, the present invention provides for a transgenic mouse having a genome comprising a homozygous disruption in its neurocalcin y̅ gene, and the disruption resulting in at least one phenotype selected from a group consisting essentially of: malfunction of sensory neurons; malfunction of normal fertility; learning disabilities; loss of memory; degeneration of neurons in the brain and combinations thereof.

In another further embodiment, the transgenic mouse possesses all of following phenotype: malfunction of sensory neurons; malfunction of normal fertility; learning disabilities; loss of memory; and degeneration of neurons in the brain.

In yet another further embodiment, the present invention relates to a method of measuring the affect of a pharmaceutical compound on neurocalcin y̅ deficiency, said method comprising: providing said compound to the mouse and measuring the affect of said compound on at least one phenotype selected from a group consisting essentially of: malfunction of sensory neurons; malfunction of normal fertility; learning disabilities; loss of memory; degeneration of neurons in the brain and combinations thereof. For purposes of this invention, the pharmaceutical compound may be any compound that can assist in the measurement of neurocalcin y̅ deficiency in a specimen.

### BRIEF DESCRIPTION OF THE DRAWINGS

The accompanying drawings are included to provide a further understanding of the present invention. These drawings are incorporated in and constitute a part of this specification, illustrate one or more embodiments of the present invention, and together with the description, serve to explain the principles of the present invention.

FIG. 1 is an image of the targeted exon-intron structure of the neurocalcin δ gene located in mouse specimen chromosome 15;

FIG. 2 is an image showing the disruption of the gene;

FIG. 3 is a schematic representation of the creation of the new DNA fragment; and

FIG. 4 is a screening image depicting which specimens have the desired genetic alterations.

Among those benefits and improvements that have been disclosed, other objects and advantages of this invention will become apparent from the following description taken in conjunction with the accompanying drawings. The drawings constitute a part of this specification and include exemplary embodiments of the present invention and illustrate various objects and features thereof.

### DETAILED DESCRIPTION OF THE INVENTION

As required, detailed embodiments of the present invention are disclosed herein; however, it is to be understood that the disclosed embodiments are merely exemplary of the invention that may be embodied in various forms. The figures are not necessarily to scale, some features may be exaggerated to show details of particular components. Therefore, specific structural and functional details disclosed herein are not to be interpreted as limiting, but merely as a basis for the claims and as a representative basis for teaching one skilled in the art to variously employ the present invention.

The present invention provides for a genetically altered specimen and related method of creating such specimen. In one embodiment, the method comprises: identifying a gene that is desired to be altered, disrupting the gene in a gene carrier to thereby create a new DNA fragment, inserting the new DNA fragment into an embryonic cell, injecting the embryonic cell which exhibits the desired genetic alteration into an embryo, inserting the embryo into a uterus of a carrier whereby the carrier's offspring shall exhibit the desired genetic alteration, and the offspring is the genetically altered animal specimen.

Neurocalcin δ is one of the several Ca²⁺-sensor components of the ROS-GC membrane guanylate cyclase transduction machinery directly linked with the sensory processes of sight, smell and taste; and in an emerging general Ca²⁺ signal transduction concept this signaling machinery is proposed to be a vital component of all the sensory and sensory-connected secondary neurons. The central theme of the concept is that “Ca²⁺ signals through a delicately controlled ROS-GC transduction machinery. ROS-GC, in turn, generates pulsated levels of cyclic GMP. Cyclic GMP then serves as a Ca²⁺ second messenger. The delicacy and specificity of the transduction machinery is achieved through its unique composition and structural design present in that particular neuron”.

The present invention will be applicable in explaining at the genetic level the molecular lesions in the sensory neurons directly linked with sight, smell and taste. The present invention will be linked with abnormalities in all of the sensory and sensory-connected secondary neurons. It will provide disease-linked gene markers, help in gene-specific therapies and explain in molecular terms functions of their expressed proteins. More specifically, the animal specimen of the present invention shall exhibit characteristics such as: the malfunction of sensory neurons, malfunction of normal fertility, learning disabilities, loss of memory, and degeneration of neurons in the brain; said characteristics of the new specimen shall be identifiable based on a protein marker for the neurocalcin δ gene.

The present invention is based upon the initiation, development, and present status of the membrane guanylate cyclase transduction field. One of these contributions is the discovery of ROS-GC transduction machinery; the demonstration that this machinery is a two-component, Ca²⁺-sensor and transduction, system; and that in addition to the photoreceptor neurons, it also exists in the secondary visual transduction neurons of the inner and outer plexiform layers (IPL and OPL) of the retina; also importantly, in the olfactory bulb neurons and its modified form in the hippocampal neurons and in the pineal gland specifically located in the pinealocytes.

One striking property of the ROS-GC transduction machinery is its elasticity for the Ca²⁺ signals generated in the sensory neurons. This property is embodied by its Ca²⁺-sensor protein partner. The present invention focuses on the partner named neurocalcin δ. Through reconstitution, mutagenesis, direct gene cloning and purification studies it has been established that neurocalcin δ is a structural part of the ROS-GC transduction system in the IPL region of the visual transduction neurons and of the ONE-GC transduction system in the odorant receptor and olfactory bulb neurons.

A very similar ROS-GC transduction system exists in the hippocampal neurons. With these considerations and the present knowledge that cyclic GMP is omnipresent intracellular second messenger of the physiological processes of sensory transduction, neural plasticity, learning and memory, cardiac vasculature, smooth muscle relaxation, blood pressure and cellular growth, the proposed animal genetic model of the present invention will form the beginning of an era where the multiple limbs of the cyclic GMP signaling pathway will be defined in the precise physiological terms and it will become possible to develop therapies for the diseases linked with these limbs of the pathway.

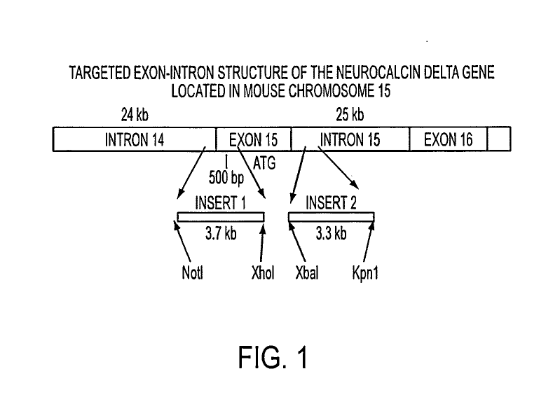

Referring now to FIG. 1, neurocalcin δ is a small calcium binding protein composed of 193 amino acid residues. In mice, the gene encoding the neurocalcin δ is located on mouse chromosome 15. The gene spans 200 kb; it consists of 16 exons and 15 introns. The coding region is constituted by exons 15 and 16. The 5′-untranslated region and 396 bp of the 5′-coding region form exon 15, 396 bp code for the first 132 amino acid residues of neurocalcin δ. The coding sequences for the remaining amino acid residues and the 3′-untranslated region form exon 16. Exons 15 and 16 are separated by intron 15 of ˜25 kb. FIG. 1 details the exon-intron structure of the targeted region of neurocalcin δ gene and the targeting vector. FIG. 1 shows the targeted exon-intron structure of the neurocalcin δ gene located in mouse specimen chromosome 15.

The present invention creates a genetically altered animal specimen using a process that knocks out a gene. To knockout a gene, a construct is engineered which should recombine with the targeted gene. The present invention accomplishes this by incorporating at least two appropriate sequences of the gene fragments into the construct. These sequences are separated by a “disruptive” sequence. The disruptive sequence is a new antibiotic resistance gene. Adding a new antibiotic resistance gene gives the present invention two advantages: 1) if properly recombined it disrupts the gene, while 2) introducing resistance to additional antibiotic allows identification of the recombinants as they become resistant to this antibiotic, whereas without recombination there will be no resistance.

There are two types of recombination of the construct with the genome, homologous (desired) and random (undesirable). Homologous recombination occurs when the construct finds the homologous sequence in the genome. This results in the insertion of a construct sequence into the gene leading to the disruption of this gene. With its sequence interrupted, the altered gene in most cases will be translated into a nonfunctional protein or not translated at all. Random recombination occurs when the construct recombines with any other site within the genome.

In order to knockout neurocalcin δ gene vector, a neomycin resistance cassette is inserted immediately after codon for the 12th amino acid residue, valine. Thus, the neurocalcin δ gene becomes disrupted and the protein neurocalcin δ is no longer properly coded. The protocol describing this procedure is shown in FIG. 1.

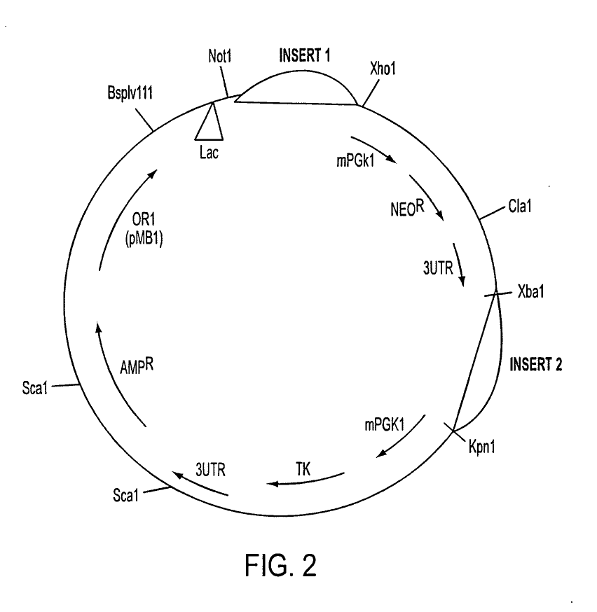

In the construct of the present invention as shown in FIG. 2, two (2) genomic fragments are amplified by polymerase chain reaction (PCR) from mouse embryonic DNA. The first fragment (INSERT 1) of 3.7 kb (chromosome 15 region 28360241-28363910) constituted part of neurocalcin δgene intron 14 and part of exon 15 encoding the 5′-untranslated region and 5′-coding region up to the codon for Val₁₂. The second fragment (INSERT 2) of 3.26 kb (chromosome 15 region 28354751-28358020) constituted part of neurocalcin δgene intron 15. The genomic distance between these two fragments was ˜1000 bp. These two fragments are cloned into pPNT vector.

The vector is digested with Not1 and Xho1 enzymes. Insert 1 is cloned into Not1/Xho1 sites of the vector). Next, the vector containing Insert 1 is digested with Xba1 and Kpn1 and Insert 2 is cloned into Xba1/Kpn1 sites. Thus, the vector contains both Inserts which are separated by the neomycin resistance gene. This strategy is shown in the lower panel of FIG. 1, above. The resulting construct was sequenced to verify proper ligation.

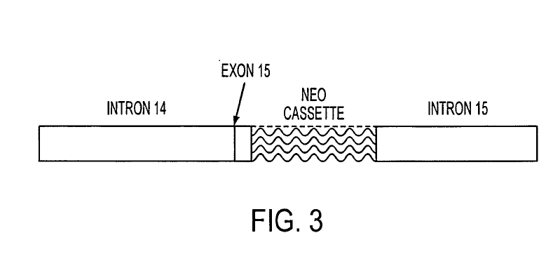

The construct is then digested with Not1 and Kpn1. As a result, a linear DNA fragment of ˜8 kb consisting of neurocalcin δgene part of intron 14, part of exon 15 encoding 5′-untranslated region and 5′-coding region starting from first ATG to amino acid 12, neomycin resistance gene and part of neurocalcin δ intron 15. The schematic representation of the linear fragment is shown in FIG. 3.

The fragment consisted of ˜3600 bp of neurocalcin δ intron 14, ˜100 bp of neurocalcin δ exon 15 encoding 5′ untranslated region and 5′-coding region starting from first ATG to amino acid Val¹², ˜1000 bp of the neomycine resistance gene and ˜3300 bp of neurocalcin δ intron 15.

In order to transform mouse embryonic stem cells for the present invention, the linear 8 kb DNA is electroporated into mouse embryonic stem cells (ES cells) growing in tissue culture. It is anticipated that some of the embryonic stem cells will pick up the DNA and the introduced DNA will hybridize with the site on chromosome 15 where the neurocalcin δ gene is located (homologous recombination).

Homologous recombination: Stretches of DNA sequence in the vector find the homologous sequences in the host genome and the region between these homologous sequences replaces the equivalent region in the host DNA.

The 8 kb DNA fragment carries the resistance to neomycin. It allows selection for any (homologous or random) recombination. If the taken up DNA recombines with the ES cells genomic DNA the cells will become resistance to neomycin. Therefore, exposing the entire culture to the antibiotic neomycin (G418) will kill cells which did not pick up the DNA, the cells however, which picked up the DNA will survive.

Not all neomycin resistant ES cells undergo homologous recombination. In fact, random recombination is predominant and only a small percent of the transfected cells will have homologous recombination.

G418 resistant clones (cell colonies) are then obtained and screened for homologous recombination. The screening involves analysis of the genomic DNA of the clones.

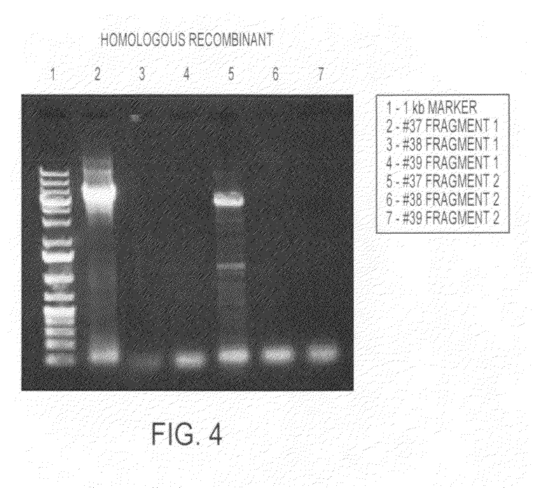

The cells are harvested and their genomic DNA isolated for screening. The DNA is subjected to PCR analysis. Using two sets of primers, two DNA fragments are amplified for each clone. A sample of data from the screening of 250 G418 resistant clones showing their genomic localizations are presented in the FIG. 4.

The first fragment amplified started in intron 14 (upstream the 5′ end of the original DNA fragment used for recombination) and finished in the neo resistance cassette; the second fragment started in neo resistance cassette and finished in the intron 15 (downstream the 3′-end of the original construct). Thus, the amplification was designed to identify only the homologous recombination. There will be no amplification of any of these fragments with random recombination.

In the sample data, one clone of ES cells with homologous recombination was identified as shown above. The clone-cells carrying the homologous recombination are then injected into mouse blastocytes and the embryos are transferred into the uterus of pseudo-pregnant mice. Among the born pups only a small percentage will carry the neurocalcin δ knockout gene mutation. The females are mated with male C-57 black mice. The offspring are genotyped and the heterozygotous offspring will be identified. After reaching sexual maturity the heterozygotes from different mothers are mated. The offspring that are produced will be screened to isolate the homozygotes.

To establish the neurocalcin δ gene knockout colony for the present invention, the identified male and female homozygote mice are mated to establish a colony of neurocalcin δ gene knockout mice.

Numerous modifications and variations of the present invention are possible in light of the above teachings. It is therefore to be understood that within the scope of the attendant claims attached hereto, this invention may be practiced otherwise than as specifically disclosed herein.

What is claimed is:

1. A genetically altered animal specimen created by a process comprising:
identifying a gene that is desired to be altered;
disrupting said gene in a gene carrier to thereby create a new DNA fragment;
inserting said new DNA fragment into an embryonic cell;
injecting said embryonic cell which exhibits the desired genetic alteration into an embryo; and
inserting said embryo into a uterus of a carrier whereby said carrier's offspring shall exhibit the desired genetic alteration, said offspring being said genetically altered animal specimen.

2. The specimen of claim 1 where said specimen is without a neurocalcin δ gene.

3. The specimen of claim 1 wherein said disruption step is performed using a construction to disrupt said gene and create said DNA fragment.

4. The specimen of claim 1 wherein said insertion of said DNA fragment into said embryonic cell utilizes electroporation.

5. The specimen of claim 1 wherein said process further comprising: mating female of said offspring from said embryo which exhibits said desired genetic alteration with male specimen with normal genetic makeup.

6. The specimen of claim 5 wherein said process further comprising: mating male and female offspring which exhibit said desired genetic alterations from different mothers to thereby create a colony of specimens exhibiting said desired genetic alterations.

7. The specimen of claim 1 wherein said animal is a mouse.

8. The specimen of claim 5 or 6 therein said desired genetic alteration is a specimen without the neurocalcin δ gene.

9. The specimen of claim 1 where said animal specimen exhibits characteristics comprising: the malfunction of sensory neurons; malfunction of normal fertility; learning disabilities; loss of memory; degeneration of neurons in the brain; said characteristics of the new specimen shall be identifiable based on a protein marker for the neurocalcin δ gene.

10. A genetically altered specimen created by a method comprising: eliminating the neurocalcin δ gene thereby having: malfunction of sensory neurons; malfunction of normal fertility; learning disabilities; loss of memory; degeneration of neurons in the brain; and a protein marker for the neurocalcin δ gene identifying said characteristics of the new specimen.

11. The specimen of claim 10 is an animal.

12. The specimen of claim 11 is a mammal.

13. The specimen of claim 12 is a mouse.

14. A process of creating a genetically altered specimen, said process comprising:
altering a selected gene in a gene carrier to thereby create a desired DNA fragment;
inserting said desired DNA fragment into an embryonic cell;
introducing said embryonic cell which exhibits the desired genetic alteration into an embryo; and
impregnating said carrier with said embryo whereby an offspring of said carrier is said genetically altered specimen.

15. The process of claim 14 wherein said specimen is without the neurocalcin δ gene.

16. The process of claim 14 wherein said alteration stage is performed using a construction to disrupt said gene and create said DNA fragment.

17. The process of claim 14 wherein said insertion of said DNA fragment into said embryonic cell utilizes electroporation.

18. The process of claim 14 wherein female of said offspring from said embryo which exhibits said desired genetic alteration is mated with male specimen with normal genetic makeup.

19. The process of claim 18 wherein male and female offspring which exhibit said desired genetic alterations from different mothers are mated to thereby create a colony of specimens exhibiting said desired genetic alterations.

20. The process of claim 14 wherein said specimen is a mouse.

21. A transgenic mouse having a genome comprising a homozygous disruption in its neurocalcin y̅ gene, said disruption resulting in at least one phenotype selected from a group consisting essentially of: malfunction of sensory neurons; malfunction of normal fertility; learning disabilities; loss of memory; degeneration of neurons in the brain and combinations thereof.

22. The transgenic mouse of claim 21 wherein said transgenic mouse possesses all of following phenotype: malfunction of sensory neurons; malfunction of normal fertility; learning disabilities; loss of memory; and degeneration of neurons in the brain.

23. A transgenic mouse of claim 21 further comprising a method of measuring the affect of a pharmaceutical compound on neurocalcin y̅ deficiency in said mouse, said method comprising: providing said compound to the mouse and measuring the affect of said compound on at least one phenotype selected from a group consisting essentially of: malfunction of sensory neurons; malfunction of normal fertility; learning disabilities; loss of memory; degeneration of neurons in the brain and combinations thereof.
